(19) United States
(12) Patent Application Publication
(10) Pub. No.: US 2002/0081330 A1
(43) Pub. Date: Jun. 27, 2002

(54) Liquid filled capsules

(76) Inventor: Victor Morrison Young, West Lothian (GB)

Correspondence Address: MYERS BIGEL SIBLEY & SAJOVEC, PO BOX 37428, RALEIGH, NC, 27627, US

(21) Appl. No.: 10/007,559

(22) Filed: Nov. 8, 2001

(30) Foreign Application Priority Data
Nov. 16, 2000 (GB) GB0027954.7

(51) Int. Cl. A61K 9/48

(52) U.S. Cl. 424/451

(57) ABSTRACT

Capsules are filled with liquid-fill by introducing the liquid into a capsule body held in an upright orientation. A capsule cap is fitted over an open end of the capsule body to close the capsule. The closed capsule is held in the upright orientation until the contents have stabilised. The holding period allows excess pneumatic pressure to be dissipated prior to sealing the cap to the body. The holding period may alternatively allow solidification of the liquid-fill. Advantageously, the closed capsules are held in an upright tube (4,6) in a vertical stack.

The present invention relates to a process and apparatus for filling a capsule with a liquid and particularly to the avoidance of problems relating to stabilisation of the contents of the capsule, especially air pressurisation within the capsule.

Capsules constitute one of the principal dosage forms for medicinal, pharmaceutical and health food products. Generally, so-called hard capsules are made from gelatin, hydroxypropylmethyl cellulose (HPMC) or other suitable material and are filled on purpose-built high speed filling machines. The capsules may be filled with materials such as powders, granules, pellets, other capsules, liquids, semi-solids or thermosetting materials.

Empty capsules are generally supplied to the filling machine in a “prelocked” condition, wherein the capsule body has a cap which is loosely attached thereto. Generally, a series of rings or protrusions are provided in the mating surfaces of the cap or body which enable the cap to be loosely attached to the body so that the cap and body are held together during storage but enabling the cap to be removed prior to filling of the capsule. Once the capsule has been filled, the cap is replaced and forced beyond the prelocked position into a fully locked position ready for sealing. Alternatively, other types of capsule filling machines are designed to accept separate supplies of capsule bodies and caps.

The capsules are closed at high speed after filling and, although most have some form of air vent in their cap or body design, this may not be totally effective at normal filling speeds in eliminating the trapping of air or other gas within the capsule, thereby leaving the filled capsule in a pressurised state (e.g. up to 1 bar) until the pressure equilibrates with the exterior.

During closure of the capsule, the cap is fitted over the body and the body is pushed up until it locks on the cap. The cap is close fitting and normally approximately half the length of the body, so it travels for a considerable distance down the capsule body before locking. This has the effect of a piston in trapping and pressurising the capsule. The excess gas normally escapes through the gap between the cap and the body, and vents may be provided in this region so as to facilitate the escape of excess pressure. When the capsule is filled with a powder fill, this trapped gas (normally air or nitrogen) is distributed throughout the spaces between the powder particles and also in the space above the fill in the body. The escape of excess gas occurs without difficulty in the case of powder-filled capsules but can be problematical with liquid-filled capsules. Thus, liquid-filled capsules have no space within the fill to distribute excess pressure, so that all gas trapped by the piston action of the cap is compressed into the small space between the liquid surface and the cap. This results in a greater pressurisation in liquid-filled capsules.

The liquid itself may also impede the release of excess pressure. Thus, when the filled capsule is ejected from the filling machine, it tumbles end-over-end and distributes the liquid fill around the interior surfaces of the capsule. Liquid tends to be drawn by capillary action between the capsule cap and body, thereby preventing or impeding release of excess pressure. The trapped internal pressure may then cause problems at the ejection stage or during subsequent sealing. If the pressure is high enough, the excess pressure may cause the cap to pop off after closing the capsule.

Alternatively, the excess pressure can cause problems during the subsequent sealing of the liquid-filled capsule. Thus, it is conventional to seal liquid-filled capsules by applying a band of polymer solution around the junction between the cap and the body. The polymer solution is generally a solution of the same polymer as the capsule cap or body in a solvent therefor. Thus, application of the solution tends to soften or partially dissolve the cap and body polymer thereby weakening them. The excess pressure can then cause deformation of the capsule. This is less a problem with gelatin capsules since they have some natural elasticity. It is a particular problem with HPMC capsules which are becoming more widely used, since they avoid gelatin (which is an animal-derived product).

A further problem which arises due to the ejection of liquid-filled capsules arises in the case of molten or thixotropic materials that take up a fixed shape in the capsule prior to administration to a patient. Thus, where the capsule is filled with a molten liquid which sets to a solid state prior to administration, it is important that the solid state be in a predetermined shape so as to provide a predictable release profile for the pharmaceutically active agent contained therein.

It is an object of the present invention to mitigate these problems.

The present invention provides a process for filling a capsule with a liquid, which comprises

introducing a liquid into a capsule body held in an upright orientation;

fitting a capsule cap over an open end of the capsule body to close the capsule; and

holding the closed capsule in the upright orientation until substantial stabilisation of the contents of the closed capsule;

said holding being completed prior to applying any sealing material to seal the capsule cap to the body.

The holding period allows stabilisation of the capsule contents (i.e. the liquid fill and trapped gas). Time is allowed for the pneumatic pressure of the gas to be released. Alternatively, time is allowed for desired stabilisation of the liquid fill, such as setting in the case of a thermosetting liquid, cross-linking etc.

In the case of release of pneumatic pressure, the closed capsule is held in the upright orientation to prevent liquid splashing around the interior of the capsule until substantial release of pneumatic pressure from within the capsule. “Substantial release of pneumatic pressure” means that pneumatic pressure is released to an extent necessary for successful subsequent processing of the filled capsule, and particularly for sealing thereof. The term “pneumatic pressure” includes air pressure and also pressure of any other gas employed in the filling process, such as inert gases including nitrogen.

The process may be operated in conventional high speed capsule filling machines well known to the skilled man, but with the provision of a means for performing the holding operation.

The invention is particularly applicable to the filling of HPMC capsules with a liquid (e.g. a vegetable oil, such as soya oil or fractionated coconut oil. Usually, the capsule is filled to the extent that 70-95%, and particularly 80-95% and generally about 90% of the body itself is filled with liquid prior to fitting the cap.

Holding is continued until substantial removal of undesired excess pneumatic pressure (or solidification, as the case may be). This generally involves a holding time in the region 5-300 seconds, particularly 10-100 seconds depending on the type of capsule employed and in particular the provision or not of any facility, such as a vent, for releasing the excess pneumatic pressure.

The liquid-filled closed capsule is generally sealed, depending on the nature of the liquid-fill and the purpose therefore. However, in some cases no sealing is required. Capsules containing a thermosetting liquid are usually left unsealed, unless they are to be coated (e.g. enteric-coated). Where a sealing material is applied to seal the cap to the body, this sealing material is applied after the capsule has been held in the upright orientation for the required length of time.

The liquid-fill may be a liquid at ambient temperatures or may be a thermoplastic or thermosetting or other material which is solid at ambient temperatures but which is filled into the capsule in a molten state (e.g. a polyethylene glycol such as PEG 6000 or PEG 8000). In this case, the capsule may be held in the upright orientation for a sufficient time to release excess pneumatic pressure and/or to allow solidification of the liquid fill. Liquid fills which solidify due to chemical reaction e.g. curing or cross-linking, are also included. The liquid-fill may be a thixotropic material which is liquid under shear pressure but which forms a gel-like mass once in place within the capsule body.

The capsule may also have a combination of fills. For example, the body may be filled with a first fill of a molten solidifying material. Once solid, a further liquid may be applied over the solid mass prior to closure with the cap. This enables complex dosage forms to be provided, allowing for two or more stage release of pharmaceutical material.

Another aspect of the invention provides an apparatus for filling the capsule with a liquid which comprises means for holding the closed capsule in the upright orientation until substantial removal of any excess pneumatic pressure inside the closed capsule. The holding means may be provided in any number of ways apparent to the skilled man, such as a carousel or series of vertical tubes.

In a particularly preferred embodiment, the closed capsules are held in a vertical array one above the other in a vertical tube. Particularly, the closed capsules are ejected from the final filling stage into the lower end of a substantially upright tube. Preferably, non-return means are provided for preventing the bottom-most capsule falling back into the ejection station. This is generally a spring-loaded or otherwise resilient means, such as a ring or detent formed of an elastomeric material. Such upright tube arrangement may be readily fitted to an existing filling machine so that the filled capsules are pushed in an upwards direction throughout the holding period wherein excess pneumatic pressure is released. The equilibrated capsules are finally ejected out of the top of the tube and are then directed back down into a conventional capsule collection area before being passed to a sealing station, if required.

Thus, the provision of a holding period enables the problem of excess pneumatic pressure generated in high speed filling machines to be alleviated in a particularly simple manner. This allows thin-walled or inelastic capsules, such as HPMC capsules, which are particularly susceptible to deformation during the sealing process to be sealed with confidence.

An embodiment of the present invention will now be described by way of example only in conjunction with the attached drawing wherein:

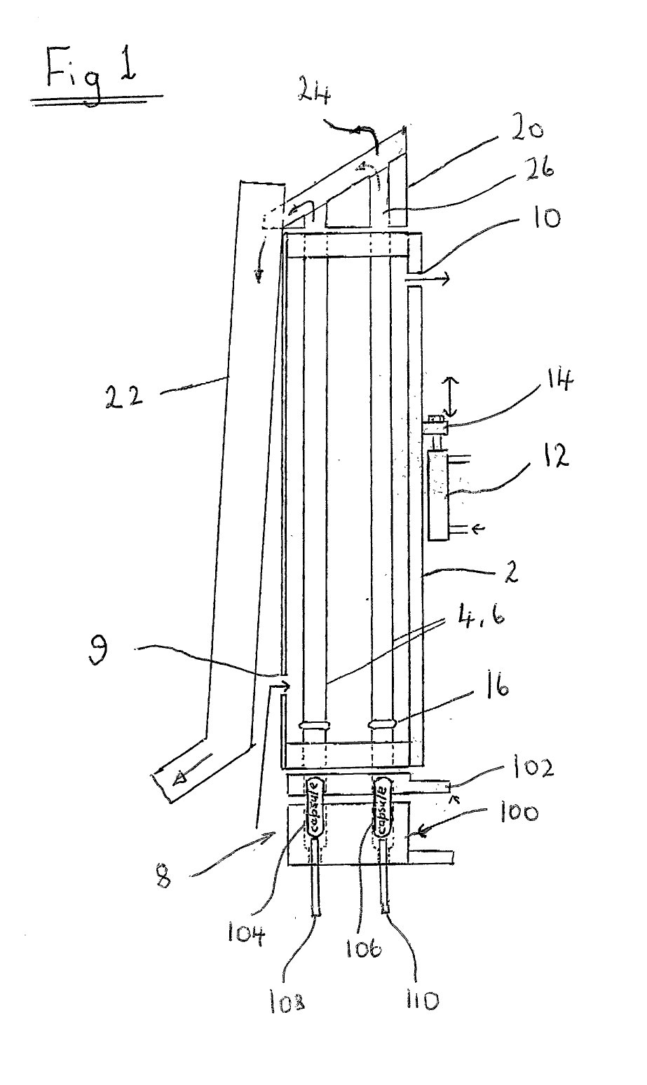

FIG. 1 is a schematic side elevation of a tube assembly for attachment to a conventional filling machine, for providing the holding period according to the present invention.

The apparatus for providing a holding period comprises an enclosure 2 containing a series of hollow substantially vertical tubes 4,6 which is arranged over the ejection station generally indicated as reference numeral 8 of a conventional filling machine.

A conventional filling machine comprises a lower block 100 for holding a batch (for example 12) of capsule bodies and a corresponding upper block 102 for holding the respective capsule caps. Typical capsules are indicated as 104,106. Ejection pins 108,110 are vertically moveable within the block 100 for vertically ejecting the filled closed capsules. According to the present invention, these filled closed capsules are ejected into the lower end of tubes 4,6 wherein they form a vertical stack. One capsule after another is ejected into the lower end of the stack until it passes out of the top thereof.

The enclosure 2 comprises an inlet 9 and outlet 10 for passing cooling gas or liquid around the upright tubes for cooling them (particularly in the case of a molten liquid fill).

A pneumatic-operated piston arrangement 12 is attached to a bracket 14 on the enclosure. The piston is arranged to lift the enclosure to enable removal of rejected capsules. It is known that conventional filling machines have a mechanism for diverting rejected capsules. This pneumatic arrangement thus operates to prevent reject capsules from entering the upright tubes.

At the lower end of each tube are provided rubber retaining rings 16 for preventing the lowermost capsule falling back towards the ejection station. These may be in the form of a groove around the outside of the tube having a slot communicating with the interior of the tube across which the rubber ring is stretched. This constitutes a resilient detent over which each capsule must pass as it is ejected into the lowermost part of the tube.

At the upper end of the tubes is provided an ejection block 20 which serves to direct the capsules ejected from the top of the tubes at the end of the holding period sideways into an ejected capsule chute 22. The chute directs the ejected capsules towards a conventional collection area. The capsules are then carried forward to a sealing station where a band of sealing material is applied in conventional manner. The ejection block is formed of a plastics material machined to provide conduits 26 aligned with the upright tubes and communicating with an inclined upper space 24 leading towards the ejected capsule chute.

The process is operated as follows. In a conventional machine the upper block 102 is aligned over lower block 100 and a batch of twelve prelocked capsules introduced into blocks 102/100. The bodies are drawn from the caps into lower block 100 by applying suction thereto leaving the caps in upper block 102.

The lower block 100 containing the capsule bodies is then moved to a filling station wherein the bodies are filled with liquid.

The lower block is then moved to a closing station where the caps are replaced over the open end of the bodies, by moving the bodies upwards into the caps into the fully locked position. The closed locked capsules are then moved to the ejection station shown in FIG. 1. At the ejection station, ejection pins 108,110 move the filled closed capsules vertically into the tubes 4,6 and past the retaining rings 16. The retaining rings prevent the capsule falling back to the ejection station. As further closed capsules are ejected into the lower end of the tubes, the tubes become filled with capsules. Each capsule is retained in the tube for the holding period until it is ejected via the ejection block 20 into the chute 22. In the case of rejected capsules, the pneumatic piston 12 is operated to raise the assembly clear of the ejection station to allow the rejected batch of capsules to be diverted elsewhere. The residence time in the tubes is generally in the region 20-40 seconds, typically around 30 seconds. This allows sufficient time for excess pneumatic pressure to be released from the liquid filled capsule prior to collection of the filled capsules and passage to the sealing station where a band of adhesive solution is applied around the junction between the cap and the body.

1. Process for filling a capsule with a liquid which comprises;
introducing a liquid into a capsule body held in an upright orientation;
fitting a capsule cap over an open end of the capsule body to close the capsule; and
holding the closed capsule in the upright orientation until substantial stabilisation of the contents of the closed capsule;
said holding being completed prior to applying any sealing material to seal the capsule cap to the body.

2. Process according to claim 1 wherein said stabilisation enables substantial release of pneumatic pressure from within the closed capsule.

3. Process according to claim 1 wherein said stabilisation enables substantial solidification of a solidifiable liquid-fill.

4. Process according to claim 3 wherein the solidifiable liquid-fill is a thermoplastic or thermosetting material introduced into the capsule body in the liquid state.

5. Process according to claim 3 wherein the solidifiable liquid-fill is a thixotropic material which forms a gel-like mass once in place within the capsule body.

6. Process according to claim 1 wherein 80-95% of the capsule body is filled with the liquid fill.

7. Process according to claim 1 wherein said holding period is in the region 10-100 seconds.

8. Process according to claim 1 wherein the capsule is formed of hydroxypropylmethyl cellulose.

9. Process according to claim 1 wherein a sealing material is applied to seal the cap to the body.

10. Apparatus for filling a capsule with a liquid which comprises;
introducing means for introducing the liquid into a capsule body held in an upright orientation;
fitting means for fitting a capsule cap over an open end of the capsule body to close the capsule; and
holding means for holding the closed capsule in the upright orientation until substantial stabilisation of the contents of the closed capsule;
said holding being completed prior to applying any sealing material to seal the capsule cap to the body.

11. Apparatus according to claim 10 wherein the closed capsules are held in a vertical array, one above the other.

12. Apparatus according to claim 10 wherein said holding period is in the region 10-100 seconds.

13. Apparatus according to claim 11 wherein the closed capsules are ejected into the lower end of a substantially upright tube.

14. Apparatus according to claim 13 wherein non-return means are provided for preventing the bottom-most capsule falling back.

15. Apparatus according to claim 13 wherein the capsules are ejected out of the top of the upright tube at the end of the holding period.
